(19) United States
(12) Patent Application Publication
(10) Pub. No.: US 2021/0137728 A1
(43) Pub. Date: May 13, 2021

(54) Wearable Back Pad for Use with Topical Pain Relief

(71) Applicant: Apex Medical Device Design LLC, Lynnwood, WA (US)

(72) Inventor: Nedaa Abdul-Ghani Nasif Al Jasim, Lynnwood, WA (US)

(73) Assignee: Apex Medical Device Design LLC, Lynnwood, WA (US)

(21) Appl. No.: 17/095,693

(22) Filed: Nov. 11, 2020

Related U.S. Application Data
(60) Provisional application No. 62/933,535, filed on Nov. 11, 2019.

(51) Int. Cl. A61F 7/08; A61F 7/02; A61F 7/10

(52) U.S. Cl. CPC A61F 7/08; A61F 2007/0024; A61F 7/106; A61F 7/02

(57) ABSTRACT

A wearable back pad used to deliver targeted topical pain relief. The wearable vest or wrap product enables a person to apply topical pain-relievers right where it is needed in cases of unilateral neck or back pain or to the upper back area that is not reachable by the person's own hands. The back pad will act as a vehicle to hold and deliver the pain-relievers in the form of consumable patch or a pocket-insert that is placed by the users themselves in the specified area with the goal of maintaining the person's independence and decreasing the need for helping hands. Back pad consumables can hold and deliver anti-itch and skin care preparations to the back hence expanding this product's indication for use.

### CROSS-REFERENCE TO RELATED APPLICATIONS

This application claims the benefit under 35 U.S.C. § 119 of Provisional Application Ser. No. 62/933,535, filed on Nov. 11, 2019, which is incorporated herein by reference.

### STATEMENT REGARDING FEDERALLY SPONSORED RESEARCH

Not applicable.

### BACKGROUND OF THE INVENTION

The back of the human torso might be affected by several painful ailments involving the muscles or the joints. Often, the pain affects only one side of the torso or is located in a specific location. Current topical therapy available for these ailments includes cloth wraps, adhesive patches, and vests that house active pain-relieving ingredients.

### Pain-Relieving Wraps:

As the name implies, wrap is a piece of cloth that is long enough to go around the lower back (hip) and is secured in place with a Velcro® strap. The wrap contains active heat-generating or cold-gel ingredients and is kept in place for a period of time, such as few hours, to provide optimal temperature to the “affected area”. Wraps contain a manufacturer pre-selected heat-generating ingredients or cold gel. Wraps are designed to be worn under clothing and may come loose during normal physical activity. Wraps can also be used for knee pain; ankle, wrist, neck and shoulder pain; and to ease menstrual cramp (menstrual heat wrap).

Currently available wraps house the pain-relievers in bilateral symmetrical distribution, hence treating both the painful and the healthy sides of the torso.

One examples of a currently available wrap includes the ThermaCare HeatWrap. This wrap is a pain-relieving over-the-counter single-use product for personal use. As depicted in FIG. 1, the cloth wrap houses several small ThermaCare heat cell discs made of a patented formula based on iron powder that is oxidized when exposed to air. When the discs are exposed it provides heat directly where the heatwrap is applied. FIG. 1 demonstrates ThermaCare back-pain therapy, and FIG. 2 demonstrates ThermaCare neck pain therapy wraps, both of which are designed to provide heat disc placement in bilateral symmetrical location.

Other examples include: CVS Health Lumbar Wrap, CryoMax Cold Therapy Ice Pack, Deep-Heat Therapy Pad, Elastoplast Heat Therapeutic Pad, Lumbar Support Belt, Back/Hip Cold Therapy Pain Relief Wrap. FIG. 3 demonstrates the Nano Cool Therapy Pain Relief Wrap with the active ingredient placed over the entire area of the wrap.

### Pain-Relieving Vests:

Current Nano Cool Therapy products includes a Nano Vest cool therapy pain relief wrap. The Nano ingredient is a patent pending alkane hydrocarbon wax placed inside clear pockets. As demonstrated in FIG. 4, the Nano Vest is designed with the clear pockets placed over the whole vest. Therefore, Nano vest cools the back on the painful and healthy side, from neck to bottom, no matter where the pain area is located.

Conversely, vests can also apply heat. In one example, an electrically heated vest heats the back from neck to bottom, no matter where the pain area is located.

### Pain-Relieving Patches:

Current patches are few inches-long rectangle, oblong, or circular patch made of an adhesive cloth which contains a manufacturer pre-selected heat-generating materials or cold gel. The patch front side is adhesive, contains the pain-reliever (active side), and adheres to the skin. Patches are over-the-counter products for personal use, applied by the person themselves onto the affected area when it is within the reach of the user hands.

Example of commercially available patches are: ThermaCare Patch, BenGay Pain Relief Patch, IcyHot Medicated Patch, HealthWise Medicated Hot Cold Menthol Patch, Hisamitsu Salonpas Hot Pain Relieving Gel Patch, Menthol Pain-relieving Patch, Hot Capsicum Plaster, Sumifun Capsicum Plaster Hot Pain-Relieving Patch, Tiger Balm patch, Rapid CBD Relief Patch, and FIR Patch that is embedded with a blend of herbs, magnetic and natural infrared bio-ceramic. The latest patches to enter the market are LUMINAS “Energy Medicine Patch” that contains 200+of mother's natures pain relievers in each patch; reflecting how current solutions are unsatisfactory, and that back-pain market have high unmet needs.

Currently available products have a multitude of limitations built in their design:

(A) Unilateral pain is treated with bilateral therapy: As seen in FIGS. 1-3, when a wrap is worn, the active ingredients are applied bilaterally to the painful and healthy sides. As an example, if the right lower back is painful the pain-relieving ingredient is applied to both right and left lower back simultaneously.

(B) Wraps are not designed for upper back therapy: Indications for the use of current wraps doesn't include upper back. If a person wanted to use a wrap for the upper back he/she needs to wear the wrap diagonally (i.e., above the right shoulder and below the left armpit) that will makes the wrap very loose, mobile, with the pain-reliever unlikely to sit close to the skin of the pain area and hence decrease its effectiveness.

(C) Patches are not designed for placement in the upper back for simple anatomical reasons; the upper back is outside the reach of the persons own hands. FIGS. 7 and 8 depict the anatomical fact that the upper back of the torso above the waistline is outside the reach of the person's own hands. Sticking a patch to a pain area above the waistline by the patients themselves is a difficult task and often requires assistance.

(D) Patches adhered to the skin can cause irritation, redness, and itching after use and can be painful to remove.

(E) As seen in FIG. 4, current pain-relieving vests, such as the Nano Vest cool therapy pain relief wrap, will cool all the user's back from neck to bottom irrespective of the location of the pain area.

(F) Pain-relieving ingredients are manufacturer pre-selected: All the currently available wraps, patches and vests are homing a manufacturer pre-selected active ingredient. Therefore, the user has no choice to use a pain-reliver that he wants as BENGAY cream, Voltaren Emogel, Biofreeze Pain Gel, Vicks Vaporub, and others known in the prior art.

### BRIEF SUMMARY

The invention relates to the field of human health aids specifically a back pad designed to act as a vehicle to hold and deliver pain-relievers, anti-allergy, cosmetic, and life style products right where it is needed.

In one embodiment, the invention comprises a back pad wrap or vest designed with a goal of unilateral application of pain-relievers to the lower back or the neck to specifically target the affected area. The pain-relievers are either applied to the wrap or vest during the manufacturing process known in the art; or applied by the person himself in the form of an adhesive patch, pocket-insert, or directly placed on a plain wrap.

In another embodiment, the invention comprises a plain back pad vest designed to mimic the shape and anatomy of the upper back of a human torso above the waistline aiming at ideal placement of pain-relievers on the upper back to target pain area outside consumer's hands reach. For this task consumable pocket-inserts and adhesive patches impregnated with the pain-relievers are supplied with the back pad, thus enabling the users to care for themselves, that he/she might not otherwise be able to do well or at all, and maintaining the person's independence and decrease the need for helping hands.

In yet another embodiment, the invention comprises a plain back pad vest designed to mimic the shape and anatomy of the back of a human torso to be used as a vehicle intended to home anti-allergy, cosmetic, skin care, and lifestyle products known in the art by designing consumable pocket-inserts and adhesive patches that home these products for topical use in the back of his/her torso. These consumables may be combined in a variety of ways that allow consumers to choose how the pieces go together. This flexibility allows the client mix-and-match options to create a semi-custom final product.

### BRIEF DESCRIPTION OF THE SEVERAL VIEWS OF THE DRAWINGS

FIG. 1 shows a back-pain therapy wrap known in the art.

FIG. 2 shows a neck pain therapy wrap known in the art.

FIG. 3 shows a cool therapy pain relief wrap known in the art.

FIG. 4 shows a cool therapy pain relief vest known in the art.

FIG. 5 depicts a back pad wrap for unilateral back pain therapy, according to one embodiment of the invention.

FIG. 6 depicts a back pad wrap for unilateral neck pain therapy according to an alternative embodiment.

FIGS. 7 and 8 demonstrate the limited reach of a person's own hands to the painful areas above the waistline.

FIG. 9 depicts the bony landmarks of the back of the human torso.

FIG. 10 depicts a frontal view of the back pad vest wrap, according to one embodiment, with an outline that simulates the back of the torso with bony markings, left and right text, and straps.

FIG. 11 depicts a frontal view of a back pad vest designed for upper back therapy.

FIG. 12 depicts a frontal view of a back pad vest designed for upper back therapy with a sailor collar.

FIG. 13 depicts a frontal view of a back pad vest demonstrating pockets distributed over the whole vest to receive, hold and deliver skin care, cosmetic, lifestyle and anti-itch preparations.

FIG. 14 depicts a user-friendly sailor collar design according to one embodiment.

FIG. 15 demonstrates the back pad hanging strap and hanging ring.

FIGS. 16A-16E demonstrate different types of consumable pocket inserts or adhesive patches for use with the back pad of the current invention, where FIG. 16A depicts a plain square insert or patch; FIG. 16B depicts a plain oblong insert or patch; FIG. 16C depicts a plain circular insert or patch; FIG. 16D depicts square insert or patch with temporary tattoo design; FIG. 16E depicts square insert or patch with attached discs.

### DETAILED DESCRIPTION

The invention relates to the field of human health aids specifically a back pad designed to act as a vehicle to hold pain-relievers, anti-allergy, cosmetic and lifestyle products and assist a person in applying these products right where it is needed.

### Definitions:

Upper back: The area of the back of human torso above the waistline.

Lower back: The area of the back of human torso below the waistline.

Unilateral: One side is affected, either right or left.

Bilateral: Both sides are affected, left and right.

Hand: refers to the upper limb or the arm in addition to the hand.

Vehicle: Is a mean for holding ingredients and placing them precisely on the pain area.

Pain-relievers: Ingredients intended for topical use to relieve pain in a particular area of the human back as known in the art, including, but not limited to, heat generating, cold applications, cool products, formula, preparations, ointment, cream, gel, heat discs, wax, and others known in the art.

Active ingredient: Include any pain-relieving, cosmetic, skin care, lifestyle, and anti-itch and anti-allergy products for topical use.

Active side of the patch: is the side that contains the active ingredients, this term will also be used for the pocket-insert.

Pocket-insert: A type of consumables for use with the back pad of the current invention which is either plain or impregnated with the active ingredients to be inserted into the vest 3 pockets.

Adhesive patch: A second type of consumables for use with the back pad having an active front side that contains the active ingredients and an adhesive back side that stick to the vest 3, away from the skin.

Front surface: Refers to the vest 3 surface of the back pad that will be in direct contact with the skin, and to which the pockets and patches are placed on.

Why treating the healthy side?

FIG. 5 shows a wrap or back pad for unilateral therapy of unilateral pain. The back pad wrap is designed with a goal of unilateral therapy with topical pain-relievers to the lower back to specifically target the pain area (treat one side only: left or right). The aforementioned topical pain-relievers are either applied to the wrap during the manufacturing process known in the art; or is applied by the user in the form of: A—a patch impregnated with user's choice pain-reliver with its adhesive side stick onto the wrap away from the skin, B—the pain-relivers are applied via a pocket-insert impregnated with user's choice ingredient, or C—the pain-relieving ingredients directly placed on the plain wrap.

In reference to FIG. 6, the back pad wrap is designed with a goal of unilateral application of pain-relievers to the neck to precisely target the affected pain area. The aforementioned topical pain-relievers are applied to the wrap: 1—during the manufacturing process known in the art, or 2—is applied by the user: A—in the form of a patch impregnated with user's choice pain-reliver placed on the active non-adhesive side, B—the pain-relivers are applied via a pocket-insert impregnated with user's choice ingredient or C—the pain-relieving ingredients directly placed on the plain wrap.

In yet another embodiment, a plain vest 3 can serve as a vehicle that hold unilateral pain-relivers for the lower back or the neck.

In reference to FIGS. 7 and 8, the upper back that is outside the reach of a person's own hands is demonstrated with pain area that need assistance during pain-relievers application.

In another embodiment, the medical aid comprises a plain back pad designed to mimic the shape and anatomy of the back of a human torso to act as a vehicle for ideal placement of products and ingredients right where it is needed and will be described later.

In reference to FIG. 9, the bony landmarks of a human back are demonstrated including the vertebral column 1 at the end of which lies the hipline, and the shoulder blades 2. Those bony landmarks will be marked on the front surface of the back pad to guide the user in applying these products right where it is needed.

In reference to FIG. 10, the vest 3 design and the complete markings that are placed on the front side of a back pad of this invention are demonstrated, wherein the bony landmarks including the vertebral column 1 that ends at the hip bone below and shoulder blades 2 are marked. The material used to manufacture the back pad is cut like a vest 3 and is further attached to multiple straps with a Velcro or similar fastening device 4 when needed, then the vest 3 is marked with indications 5 (i.e. “Your Left” and “Your Right”) instructing the user to identify the exact location of his/her affected area that needs to be treated. This vest 3 is plain, acting as a vehicle to hold user choice of available therapeutic ingredients.

### A Back Pad for Upper Back Pain-Relieving Therapy:

In reference to FIG. 11, example of multiple pockets that are attached to the plain back pad are depicted and are placed in locations that specifically target the affected pain area in the upper back. These pockets act as receivers for inserts that contains active pain-relieving ingredients. This back pad act as a vehicle for ideal placement of topical pain-relievers on the upper back to target pain area that is outside the reach consumer's hands thus enabling an individual to care for her/himself, that he might not otherwise be able to do well or at all, and maintaining the person's independence and decrease the need for helping hands.

In reference to FIG. 12, a central pocket is placed to hold and deliver pain-relievers to pain area located at the middle of the upper back, and the vest 3 is user-friendly with a sailor collar 7.

### Back Pad Cosmetic Customization:

Expanding the indication for use of the back pad depicted in FIG. 13, is achieved by designing pocket-inserts 6 and adhesive patches 6 that hold cosmetic, skin care and lifestyle products and are used in conjunction with the plain back pad that serve as a vehicle to place them right where needed, hence facilitates wearing these products on the skin of the upper back and lower back. Examples of cosmetic, skin care and lifestyle products include, but not limited to, moisturizers, skin care, suntan, sunscreen, skin whitening products, temporary tattoo design, glitter, and perfumes.

### Back Pad for Anti-Itch, Anti-Allergy Therapy:

According to one embodiment of this invention, additional indication for use of back pad is achieved by designing pocket-inserts 6 and adhesive patches that hold anti-itch products used in conjunction with the plain Back pad that serve as a vehicle to place them right where it is needed.

### User-Friendly Back Pad:

To simplify wearing the back pad either a sailor collar 7 or a hanging ring 8 (FIG. 15) is attached to the top of the back pad.

In reference to FIG. 14, the upper straps of back pad vest 3 are replaced with a sailor collar 7 with the goal of making the back pad easier to wear and holds better in place and the sailor collar 7 might be favored by users with shoulder joint issues or poor coordination in handling the straps.

In reference to FIG. 15, a hanging strap or a hanging ring 8 is placed at the top of the back pad vest 3 that the back pad can be hanged on a wall with the front side facing towards the user to place his selected pocket-inserts 6 or patches 6 and the back pad is ready to wear.

### Back Pad Consumables: Pocket-inserts and Adhesive Patches:

In reference to FIG. 16, two types of consumables are depicted for use with the back pad of this invention pocket-inserts 6 and adhesive patches 6 that are interchangeably placed on the back pad. The inserts and patches come as either a plain material or impregnated with any pain-relieving ingredients, skin care, cosmetics, temporary tattoo design, lifestyle products or anti-itch products; that hold and deliver the product locally to the skin on the desired specific area. These consumables may be combined in a variety of ways that allows consumers to choose how these pieces go together. This flexibility allows the client mix-and-match options to create a semi-custom final product.

I. Pocket-inserts 6 are made from a plain material that is cut into few inches long sterile individually wrapped pieces of different shapes, for example a square shape as depicted in FIGS. 16A (square), FIG. 16B (round), or FIG. 16C (rectangle). The plain pocket-insert can be impregnated with any of the above-mentioned products to hold and deliver the ingredients to the skin in the desired area.

II. Adhesive Patches are made from an adhesive material that is cut into few inches' long shapes. The back pad adhesive side is advantageously designed to stick to the vest 3 or the wrap away from the skin. The non-adhesive surface contains a piece of material that can be either plain or impregnated with any of the above-mentioned products to hold and deliver the ingredients to the skin in the desired area. Further reference to FIGS. 16D-16E, a patch with a temporary tattoo design (FIG. 16D) and a patch in which the active ingredients are in the form of a heat disc, cool cell, healing magnet, healing copper, hot stones, massaging balls or similar currently available pain-relieving product (FIG. 16E).

### Additional Use for Back Pad Patches:

Currently available patches stick directly to the skin and can produce skin irritation, redness and itch that lasts for some time. In contrast, back pad patches stick to the vest 3 or the wrap of this invention. This characteristic feature can be used to avoid the skin irritation. A back pad patch can replace current patches, hold and deliver the needed active ingredients known in the art without skin irritation.

### Materials Used to Manufacture a Back Pad and its Consumables:

Any type of material known in the art is suitable for manufacturing the vest 3 or the inserts, and the type of material is not a limiting feature for the current invention. Back pad vest 3 and wrap can be made from any of the following material: any cloth or fabric, it can be stretchable, breathable, woven or non-woven fabric. It can be made from water-proof material, paper or foil, or similar material known in the art.

The material used to manufacture the back pad is cut similar to the back piece of a vest 3.

Back pad vest 3 can be strengthened along the vertebral column 1 to fit the shape and configuration of the human torso and be kept closer to the skin. The vest 3 comes in different sizes X-small to XX-Large inclusive.

The back pad pockets are preferably made of a mesh non-absorbable material that allows the impregnated preparation to be delivered to the skin of the affected area.

The back pad pocket-inserts 6 can be made of plain fabric material, gauze, cotton, paper, sponge or any other suitable material that can be impregnated (embedded, soaked or saturated) with the person's choice of cosmetic or pain-relieving cream/ointment or gel, and are suitable for placement inside the back pad pockets. The pocket-insert 6 can have a layer of water-proof material on their back surface. Each insert is sterile and individually wrapped.

The back pad adhesive patches are made of breathable stretchable no-mess plaster or fabric that is easy to apply and remove known in the art. The back side of the patch is adhesive resting on a peel off backing, the front surface is the active side that is impregnated with the pain-relieving, anti-itch ingredients, skin care, cosmetic, or lifestyle products added either during the manufacturing process or by the user similar to what have been described above in relation to pocket-insert. The patches can stick to the vest 3 or the wrap using a velcro binder 4. The patch size is known in the prior art and is not a limiting feature of the current invention.

The back pad vest 3, wrap, inserts and patches 6 are either for one-time use or are reusable.

### Method of Use of Back Pad:

The back pad is placed flat with the front surface facing upwards, then the user will select the desired pocket-inserts 6 and place them inside the pocket in the precise area in need for the therapy. The back pad is then worn by the user and the straps are tied. The back pad remains in contact with the skin according to the product recommendation.

In case the user chooses an adhesive patch for use on a plain back pad vest 3, different patches may be combined in a variety of ways that allow consumers to choose how the pieces go together. This flexibility allows the client mix-and-match options to create a semi-custom final product.

Each back pad package contains full instructions that enables the individual to use the invention in a stepwise method.

### Benefits of the Current Invention:

(1) Enabling a person to care for themselves, that they might not otherwise be able to do well or at all; (2) maintaining the person's independence and decreasing the need for helping hands; (3) Enhancing the overall wellbeing of the person; (4) Reducing the burden placed on health care givers, particularly for people with long-term health conditions; (5) Improving a person's functionality; (6) Increasing a person's choices to apply cosmetic preparations to their back; keeping with current lifestyle.

What is claimed is:

1. A back pad wrap for unilateral lower back and hip pain therapy in which any generic heat-generating ingredients, cold gel and all pain-relieving ingredients should be placed either on the left or the right half of the wrap only with the goal of selectively treating the painful side.

2. A back pad wrap for unilateral neck pain therapy in which any generic heat-generating ingredients, cold gel and all pain-relieving ingredients should be placed either on the left or the right half of the wrap only with the goal of selectively treating the painful side.

3. A plain back pad vest designed to mimic the shape and anatomy of the back of a human torso that act as a vehicle to facilitate accurate placement of the active ingredients:
Wherein the material used to manufacture the back pad is cut similar to the back piece of a vest;
Wherein the back pad vest have a front surface and a back surface;
wherein the front surface is marked with bony landmarks including the shoulder blades and the vertebral column that ends at the hip bone below;
Wherein the front surface acts as a vehicle to receive a patch or a pocket insert that hold user choice of available active ingredients including any pain-relieving, cosmetic, skin care, lifestyle and anti-itch and anti-allergy products for topical use;
Wherein the vest is marked with a text marking indicating the user left and right side instructing them to identify their affected side that needs the therapy;
wherein the vest is attached to multiple straps with a velcro as needed.

4. The plain back pad vest of claim 3 wherein multiple pockets are fixed on the vest front surface:
Wherein the pockets are intended to receive and hold the pocket-inserts.
Wherein the number of the pockets attached to the vest is variable;
Wherein the location of the pockets is variable;
Wherein the size of the pockets is variable.

5. The pocket-inserts of claim 4 wherein the pocket-inserts are a piece of material that can be inserted inside the back pad vest pockets;
Wherein the pocket-insert can hold and deliver the active ingredients including any pain-relieving, cosmetic, skin care, lifestyle and anti-itch and anti-allergy products for topical use;
Wherein the active ingredients are impregnated and attached during the manufacturing process;
Wherein the active ingredients are placed by the consumer;
Wherein multiple pocket-inserts are supplied as sterile individually wrapped consumables.

6. The plain back pad vest of claim 3 wherein the adhesive patches are a piece of material that can stick to the back pad vest front surface:
Wherein the patch has two sides one adherent and second active surface that carries the active ingredients;
Wherein the adhesive side is placed on the back pad front surface away from the skin;
Wherein the active side can be impregnated with any active ingredients including any pain-relieving, cosmetic, skin care, lifestyle and anti-itch and anti-allergy products for topical use;
Wherein the active ingredients are impregnated and attached during the manufacturing process;
Wherein the active ingredients are placed by the consumer;
Wherein multiple adhesive patches are supplied as sterile individually wrapped consumables.
Wherein the patches can be used singular or multiple;
Wherein multiple patches be combined in a variety of ways that allow consumers to choose how the pieces go together. This flexibility allows the client mix-and-match options to create a semi-custom final product.

7. The plain back pad vest of claim 3, wherein the vest is customized to act as a vehicle to facilitate accurate placement of pain-relieving therapy on the upper-back to target pain area that is outside the reach consumer's hands wherein the pockets and patches are exclusively placed above the waistline on the center and on the sides.

8. The plain back pad of claim 3 The plain Back pad of claim 3 where in the upper straps are replaced by a sailor collar.

9. The back pad of claim 3 wherein the vest is attached to a hanging strap and hanging ring.

10. The back pad of claim 3, further comprising an adhesive patch in which the adhesive side is placed on the vest or wrap is used in persons having skin irritation, redness or itching from the current patches.

11. The back pad of claim 3, wherein the area of the vest marked by the vertebral column or the shoulder blades are thickened.
